(19) United States
(12) Patent Application Publication
(10) Pub. No.: US 2020/0410601 A1
(43) Pub. Date: Dec. 31, 2020

(54) AUTOMATED PRIOR AUTHORIZATION REQUEST GENERATION AND TRACKING

(71) Applicant: Verata Health, Inc., Edina, MN (US)

(72) Inventors: Rob Laumeyer, Bloomington, MN (US); Brent Backhaus, Sioux Falls, SD (US); Jeremy Friese, Bloomington, MN (US); Jeffrey D. Cowan, Eden Parairie, MN (US)

(21) Appl. No.: 16/912,056

(22) Filed: Jun. 25, 2020

Related U.S. Application Data
(60) Provisional application No. 62/866,040, filed on Jun. 25, 2019.

(51) Int. Cl. G06Q 40/08; G06N 20/00; G06F 40/174; G16H 10/60; G06F 40/289

(52) U.S. Cl. CPC G06Q 40/08; G06N 20/00; G06F 40/289; G16H 10/60; G06F 40/174

(57) ABSTRACT

A computer implemented method includes receiving an indication of a medical order for a patient, accessing insurance information of the patient, such insurance information identifying a payor, accessing an electronic medical record corresponding to the patient, using a machine learning program trained on properly filled out payor specific prior authorization request to generate a prior authorization request corresponding to the medical order based on data in the electronic medical record, and submitting the generated prior authorization request to the payor for processing.

### RELATED APPLICATION

This application claims priority to U.S. Provisional Application Ser. No. 62/866,040 (entitled AUTOMATED PRIOR AUTHORIZATION REQUEST GENERATION AND TRACKING, filed Jun. 25, 2019) which is incorporated herein by reference.

### BACKGROUND

Medical insurance companies may require prior authorization for certain medical treatments, use of diagnostic equipment, and for other procedures corresponding to an order created by a physician/provider. For example, the order may be for a procedure such as a knee replacement, a medication such as Harvoni, or a device such as a CPAP (Continuous Positive Airway Pressure machine).

Each insurance company may have a different process and require different information in order to approve or reject authorization, meaning payment will not be made without authorization. It can be difficult to obtain the information required and properly submit it to the insurance company or companies. Many times, various medical codes may be required that may or may not be in the created order.

### SUMMARY

A computer implemented method includes receiving an indication of a medical order for a patient, accessing insurance information of the patient, such insurance information identifying a payor, accessing an electronic medical record corresponding to the patient, using a machine learning program trained on properly filled out payor specific prior authorization request to generate a prior authorization request corresponding to the medical order based on data in the electronic medical record, and submitting the generated prior authorization request to the payor for processing.

### BRIEF DESCRIPTION OF THE DRAWINGS

FIG. 1 is a block flow diagram illustrating a computer implemented method of creating a new prior authorization request to obtain approval from an insurance company to proceed with a treatment, such as a service or procedure according to an example embodiment.

FIG. 2 is a canonical form capturing information for use in generating prior authorization requests according to an example embodiment.

FIG. 3 is a flowchart illustrating a computer implemented method of populating prior authorization requests according to an example embodiment.

FIG. 4 is a flowchart illustrating an example computer implemented method of determining whether or not the request is likely to be approved by a payor according to an example embodiment.

FIG. 5 is a block diagram of an example of an environment including a system for neural network training, according to an embodiment

FIG. 6 is a block schematic diagram of a computer system to implement one or more example embodiments.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

The functions or algorithms described herein may be implemented in software in one embodiment. The software may consist of computer executable instructions stored on computer readable media or computer readable storage device such as one or more non-transitory memories or other type of hardware-based storage devices, either local or networked. Further, such functions correspond to modules, which may be software, hardware, firmware or any combination thereof. Multiple functions may be performed in one or more modules as desired, and the embodiments described are merely examples. The software may be executed on a digital signal processor, ASIC, microprocessor, or other type of processor operating on a computer system, such as a personal computer, server or other computer system, turning such computer system into a specifically programmed machine.

The functionality can be configured to perform an operation using, for instance, software, hardware, firmware, or the like. For example, the phrase “configured to” can refer to a logic circuit structure of a hardware element that is to implement the associated functionality. The phrase “configured to” can also refer to a logic circuit structure of a hardware element that is to implement the coding design of associated functionality of firmware or software. The term “module” refers to a structural element that can be implemented using any suitable hardware (e.g., a processor, among others), software (e.g., an application, among others), firmware, or any combination of hardware, software, and firmware. The term, “logic” encompasses any functionality for performing a task. For instance, each operation illustrated in the flowcharts corresponds to logic for performing that operation. An operation can be performed using, software, hardware, firmware, or the like. The terms, “component,” “system,” and the like may refer to computer-related entities, hardware, and software in execution, firmware, or combination thereof. A component may be a process running on a processor, an object, an executable, a program, a function, a subroutine, a computer, or a combination of software and hardware. The term, “processor,” may refer to a hardware component, such as a processing unit of a computer system.

Furthermore, the claimed subject matter may be implemented as a method, apparatus, or article of manufacture using standard programming and engineering techniques to produce software, firmware, hardware, or any combination thereof to control a computing device to implement the disclosed subject matter. The term, “article of manufacture,” as used herein is intended to encompass a computer program accessible from any computer-readable storage device or media. Computer-readable storage media can include, but are not limited to, magnetic storage devices, e.g., hard disk, floppy disk, magnetic strips, optical disk, compact disk (CD), digital versatile disk (DVD), smart cards, flash memory devices, among others. In contrast, computer-readable media, i.e., not storage media, may additionally include communication media such as transmission media for wireless signals and the like.

In an Electronic Medical Record (EMR), a physician/provider creates an order for treatment for a patient. The order may be for a procedure such as a knee replacement, a medication such as Harvoni, a device such as a CPAP (Continuous Positive Airway Pressure machine), or any other type of treatment for which reimbursement may be sought for from an insurance company providing insurance for the patient.

An automated prior authorization request generating system that is programmed with software is alerted to the order by either an API (Application Programming Interface) push message or by polling new orders via a pull message using either API or HL7 (Health Level 7), or occasionally Secure Direct Messaging or Flat Files. The system practices what is referred to as “Promiscuous Data” which means that the system takes whatever data that is available, in whatever format and syntax is present, and mines the data for information suitable for requesting prior authorization. The orders entered into the EMR by the provider are placed onto the system's worklist. A worklist may also be generated for each prior authorization request to be generated, corresponding to the information required to complete the request, such as particular fields of data needed, supporting documents required, and other information as somewhat uniquely specified for each procedure or service by each different payor.

The system queries the EMR data to determine the patient's current insurance company, group/plan and policy number. This process may query discrete data from the EMR fields, or it may query the customer's document repository for a scanned copy of the patient's insurance card. If the insurance card data is a scan of the card, the system uses OCR (Optical Character Recognition) and text logic to identify the company and subscriber identification numbers. The system completes this task for both the health insurance numbers and the prescription benefit numbers.

The system then queries the insurance company based on information obtained from the card to verify current coverage. If a negative answer is returned, the system sends the query to several other insurance companies in parallel, to identify which company is currently covering the patient. If this second step fails, the worklist item is put into a queue for a person to follow up by contacting the patient and getting current information.

The provider's order is almost always specified in words. The system analyzes the words that define the order and attempts to assign a CPT (Current Procedure Terminology), HCPCS (Healthcare Common Procedure Coding System), or NCPDP (National Council for Prescription Drug Programs) code to the order. If this step fails, then the worklist item is put into a queue for a person to assess and assign the correct code.

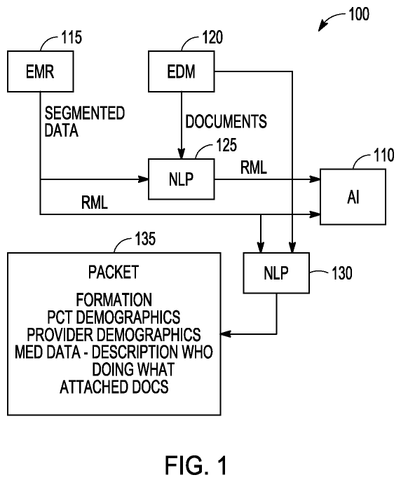

FIG. 1 is a block flow diagram illustrating a computer implemented method 100 of creating a new prior authorization request to obtain approval from an insurance company to proceed with a treatment, such as a service or procedure. An AI system 110 includes one or more models that have been trained to identify information for inclusion in a prior authorization request form related to a procedure or service for which prior authorization from an insurance company is required. The AI system 110 receives information from fields of an electronic medical record (EMR) database 115 as well as information from documents from an electronic document management (EDM) system 120. The documents may be images, PDFs, or other binary large objects (BLOBS) not amenable to being directly stored in fields of the EMR database. Such documents may be referenced in the EMR database 115 and thus a part of the patients electronic medical record.

The information may be provided to the AI system 110 directly in a canonical form or following natural language processing (NLP) 125. The canonical form (Real Medical Language (RML)) is basically a standard format that is used in the training of the AI system 110 models for consistency of information that may be received from multiple different entities involved in the treatment of patients. RML is the storage architecture and the functions/code which organizes the information. Use of a common date format, common phone number format, common address format, common name formats, etc., will ensure training data as well as data provided for processing will be consistent to provide more accurate results.

A user interface 130 provides a user with a view of data being populated in the prior authorization request as well as the ability to edit that data. A prior authorization request packet 135 is prepared based on the requirements of each insurance company, with supporting information as well as supporting documents. The supporting information may include patient demographics, provider demographics, and medical data including a description of who is performing the procedure or service, what is being done, where, and when.

The following is an example patient encounter summary preceding a recommendation for knee replacement:
“History of Present Illness
CHASE HOLLYWOOD is a 60 year old male with right knee pain which has been ongoing for several months. The pain is localized to the medial aspect of the knee and is aggravated by walking. He works as a custodian and walks about 4 miles per day while he is at work. CHASE has not had any treatment at this time for the knee.
Results/Data
CHASE had x-rays taken at Allina of the right knee that were reviewed and show diffused degenerative changes throughout the right knee
Procedure
Procedure: Injection of the right knee joint. Indication: osteoarthritis and pain. Risk, bleeding risk, benefits, infection risk, alternatives, allergic reaction risk and recurrence were discussed with the patient. Verbal consent was obtained prior to the procedure. The area was prepped using betadine, ethyl chloride spray was used as a topical anesthetic.
Using a sterile technique, the patient was injected with 4 mL of 1% Lidocaine and 2 mL of 40 mg/mL Kenalog. No epinephrine was used. Lot #'s: 8269DK/AAN68. A bandage was applied. The patient tolerated the procedure well. There were no complications.
Diagnosis
Primary osteoarthritis right knee
Discussion/Summary
CHASE and I had a detailed discussion regarding the etiology, pathophysiology and history of knee osteoarthritis and the treatment options including observation, physical therapy and a cortisone injection. CHASE has elected to proceed with right knee injection today. We will observe CHASE's symptoms and can return to clinic in three weeks for repeat evaluation or as needed.
Carbon Copy Recipients
Signatures
Electronically signed by: Jay Laumeyer, M.D.; Apr. 15 2020 1:20 PM CST
Provider Statement: I personally performed this service and the scribe documentation accurately reflects this service.”

In this example, it is assumed that the provider received a call from the patient a few weeks following the appointment summarized above indicating that this treatment was not working. The provider indicates that knee replacement surgery is required. A discussion of the implications of a surgery would be had with the patient. Notes from that call or visit are then entered into the patient's electronic medical record and an order may be generated, kicking off method 100. The order may be received by system 100 either by direct notification referred to as push, or by system 100 periodically querying the EMR 115, referred to as pull.

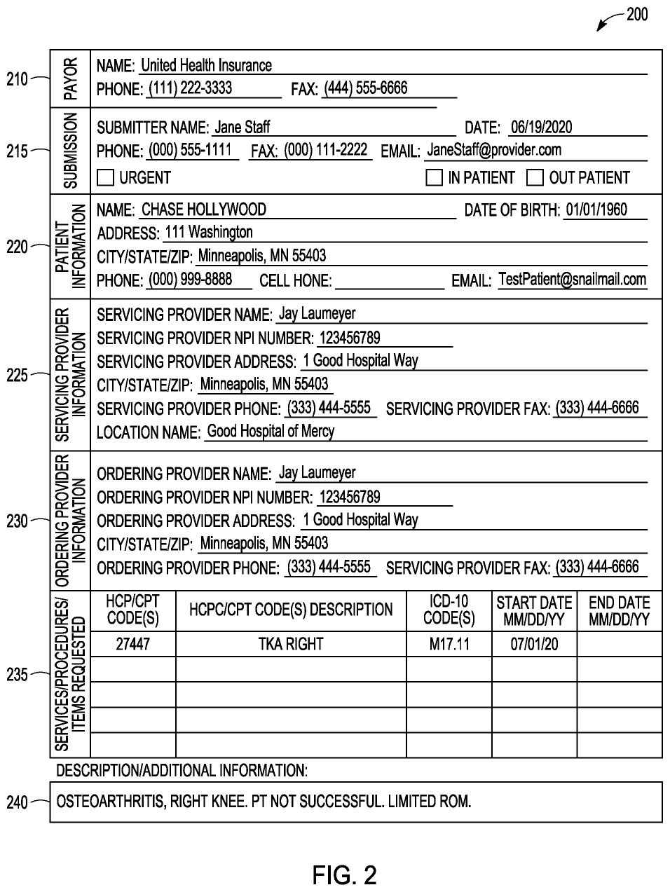

FIG. 2 is a canonical form 200 capturing information for use in generating prior authorization requests. Form 200 includes payor information 210, submission information 215, patient information 220, servicing provider information 225, ordering provider information 230, items requested information 235, including code 27447 corresponding to a TKA right knee replacement. An ICD-10 code of M17.11 is provided along with a start date of Jul. 1, 1920. A description 240 includes additional information supporting the order. In this case, the additional information identifies the condition as osteoarthritis, right knee. It is also indicated that physical therapy (PT) was not successful and that range of motion (ROM) is still limited.

In this example, much of the information may be pulled directly from the EMR database 115 as the information maps to EMR field names. Information may also or alternatively be pulled via HL7/API/FHIR/REST or direct database access. Any method of inquiry to the data are equivalent if they expose all necessary data. Extraction of data from UI of the software can also be used to acquire so of the data needed. Some information may be obtained from text analysis of the order via NLP 125, such as the description and additional information 240. Still further information, such as codes may utilize one or more AI system 110 models. The AI system 110 models may be trained using thousands of examples of orders that are labeled with correct codes. Additional models may be used for other required information that is not easily deduced from orders and other patient information using similar training examples.

Canonical form 200 may be used to populate specific forms required by each different payor in various embodiments, either offline, or in a live interactive session via network connection to a payor portal. Since each payor may have different requirements for difference procedures, and differences exist between payors in the information required and forms used, the mapping between the canonical form 200 is tailored to each payor's requirements.

In one embodiment, each payor may have medical policies that describe the information required for each procedure/service. Each such policy may be translated to the RML format such that form 200 can be used to translate data from it into the form required by the policy for preparation of the PA authorization packet. This can be tedious work and may be done by a combination of automation and human labor in various examples.

In one example medical policy for knee replacement surgery, required clinical information required may include medical notes documenting all of the following:
Specific diagnostic image(s) that show the abnormality for which surgery is being requested, which may include MRI, CT scan, X-ray, and/or bone scan; consultation with requesting surgeon may be of benefit to select the optimal images. Note: Diagnostic image(s) must be labeled with: the date taken, applicable case number obtained at time of notification, or member's name and ID number on the image(s).
Diagnostic image(s) report (s).
Condition requiring procedure
Severity of pain and details of functional disabilities interfering with activities of daily living (preparing meals, dressing, driving, walking) using a standard scale, such as the Western Ontario and McMaster Univerties Arthritis Index (WOMAC) or the Knee injury and Osteoarthritis Outcome Score (KOOS)
Physician's treatment play including pre-op discussion
Pertinent physical examination of the relevant joint.
Co-morbid medical condition(s).
Therapies tried and failed of the following including dates: Orthotics, Medications/injections, physical therapy, surgical, other pain management procedures
Date of failed previous surgery to the same joint (proximal tibial or distal femoral osteotomy, if applicable)
For revision surgery, include documentation of the complication and the complete (staged) surgical plan
For CPT codes 27446 and 27447; if the location is being requested as an inpatient stay, provide medical notes to support at least one of the following: surgery is bilateral, member has significant co-morbidities; include the list of comorbidities and current treatment; member does not have appropriate resources to support post-operative care after an outpatient procedure; include the barriers to care as an outpatient.

This particular policy may be used for each of the following CPT codes: 27445, 27446, 27447, 27486, and 27487. The particular code for the above example with CHASE HOLLYWOOD is 27447, corresponding to: Arthroplasty, knee, condyle and plateau; medial and lateral compartments with or without patella resurfacing (total knee arthroplasty).

As can be seen, the complexity of each such policy is compounded by the sheer volume of different procedures and different payors. The transformation of the EMR and EDM information into RML as well as the encoding of policies into RML provides the ability to use machine learning to help compile information needed to successfully generate prior authorization packets efficiently.

The process of creating simplified structural concepts which are then combined in an easy to understand and follow logic is solved by the structure of the RML. With more than 20,000 possible codes, 4,000 payer plans, and up to hundreds of requirements per code one can see that generation of accurate request packets greatly exceeds any human capacity for recall much less understanding. The RML canonical representation and inherent decomposition allows for meaningful representations of the unique percepts and for these to be represented a single time. This allows for a meaningful reduction in the number of columns which have to be analyzed by the neural network, which makes this a problem which can currently be solved.

A unique challenge to automation occurs frequently regarding the specification of when the order is to be performed, meaning when is the surgery to be scheduled or the echocardiogram to be measured. Often, the physician will specify “in six months” or “two weeks before the next appointment”, literally. The system 100 may employ a combination of fuzzy logic and machine learning to determine a date or date range to provide with the prior authorization request.

In one embodiment, the date may be given, such as in the following example order: “See patient for follow up June 2.” This date is used as the data of service or procedure. Fuzzy logic may be used to identify the month and a day of the month in close proximity to each other. The year may be implied based on the current known date.

In other cases, the data may be indefinite, such as “See pt f/u in 6 weeks”. To process this indefinite date, several facts may be gleaned from the data associated with the patient, such as date of the order or note, historical data of similar notes that show the length of time resulting, the specialty or class of the procedure, or even indefinite text extracted or encoded. These facts may be provided to a neural network model that has been trained on similar labeled training material to derive a date for the procedure or service and to populate the PA form. The label provides a correct date or range of dates in canonical form for each training example.

An additional unique challenge to automation arises when human users of any particular EMR place required data into the wrong fields. Such wrong field data placement may happen frequently. Since the system is not in a position to influence or incentivize how any user uses their EMR, the system 200 must sort out the data that is required, regardless of how the field is named or tagged. The system combines fuzzy logic, contextual understanding, and anticipation of wayward behavior built within compact AI 110 and NLP (Natural Language Processing) 125 algorithms to address such wrong field placement of data. The analysis of freeform data to extract data utilizes NLP and simple modeling which are tuned to look for and extract known values. These methods can range from regex looking for dates, codes or delimited data to models which look for other known entities which are free text.

With the code and the payer identified, the system accesses an internal database to find the specification regarding whether a Prior Authorization (PA) is required by the payer. If PA is not required, a worklist item is marked as No PA Required. If PA is required, then the process continues.

The database referred to in the above paragraph may be built and maintained via two methods. The first method is to scan the payer's public website to find PDF documents and other items that define the list of codes that require PA. The second method is used when a partner relationship exists between a party making or using the system and the payer, such that the payer's data is transmitted to the system on a periodic proprietary basis. This same database is used in later steps to pull the payer's PA Form required data items and the payer's medical policy information requirements, typically in the form of questions and logic trees.

The system then extracts additional information from the EMR 115, including full patient demographics as well as clinical notes and data going back several months. The patient's non-EMR data is also identified and retrieved from the site's document repository, EDM 120, including PDF and other image-type documents that were associated to the patient from faxes and scans of documents from outside providers (such as labs or imaging centers), or often from documents the patient themselves brought to the doctor.

At this stage, to analyze and decipher the clinical notes and other data contained in the EMR extraction, many vendors will implement dictionaries such as SNOMED or LOINC, but then fail due to the constraints placed on their application by using such static resources. The system 100 has combined many sources of dictionaries along with context developed by analyzing real medical language found throughout several EMRs used by many different organizations. This extended effort has captured a wide range of data formats and syntax, resulting in the ability to programmatically understand the Real Medical Language (RML).

Since many EMR systems also represent data obtained from outside sources in an ingested format in which the data was received, the system utilizes unique methods to analyze and include this additional data when necessary. As an example, many patients use Physical Therapy clinics, radiology imaging centers, and labs that are not part of, or connected with, the physician who is placing the service order. But data and results from these various other providers must be included in the Prior Authorization submission, packet 135, to the payer. Much of this third-party data is sent to the physician via fax. Many times, the patient will hand-carry stacks of paper reports, which are then scanned and stored at the physician's office. While a human user of the given EMR may find these documents via a link within the EMR screens, this raw data itself is not formally contained within the EMR. It is stored as PDF or TIF image data in a document repository such as EDM 120.

In some embodiments, the system utilizes commercially available OCR libraries to convert the PDF and TIF image data into text that can be processed. Once converted to text, the techniques described above are employed, to determine if that particular document is required to satisfy the payer's medical necessity questions in order to generate the PA.

The sum of all these components is referred to as Medical Language Processing (MLP), with the goal of consuming whatever data is available and creating an understanding of the context and completeness of this data as it is applied to satisfying the payer's requirements relative to a Prior Authorization request.

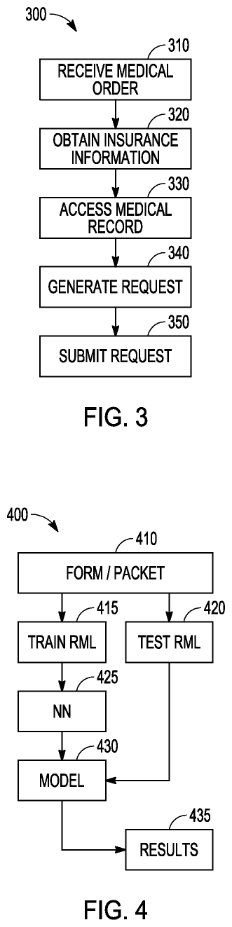

FIG. 3 is a flowchart illustrating a computer implemented method 300 of populating prior authorization requests according to an example embodiment. Method 300 begins by receiving an indication of a medical order for a patient at operation 310. Medical orders may be received by polling new orders via a pull message or by receiving push messages corresponding to medical orders. Insurance information of the patient is obtained at operation 320. The insurance information identifies a payor. A payor system may be queried to verify patient coverage. At operation 330, an electronic medical record corresponding to the patient is accessed. The medical records may be in the form of a relational database and may also include documents, such as images in the form of binary large objects.

At operation 340, a machine learning program trained on properly filled out payor specific prior authorization requests is used to generate a new prior authorization request corresponding to the medical order based on data in the electronic medical record. The generated new prior authorization request is submitted to the payor at operation 350 for processing.

In some embodiments, the electronic medical record may be queried for the new prior authorization request specific information. A combination of fuzzy logic and machine learning may be used on orders and optionally on other data to determine a date or date range from the medical order to provide with the new prior authorization request. Optical character recognition may be performed on electronic medical record data not in text form.

In some embodiments, a combination of fuzzy logic, contextual understanding, and anticipation of wayward behavior built within compact AI and NLP (Natural Language Processing) algorithms may be used to address wrong field placement of data in the electronic medical record.

A checklist identifying data items for generating the new prior authorization request may also be generated based on each payor's requirements. The checklist may be used to determine data items in the checklist not satisfied and to determine whether or not the payor will approve the new prior authorization request without the data items determined to be not satisfied. As the data is being extracted from the EMR, in parallel the system matches the payer-code combination to the list of form data and medical necessity information items in the database. The checklist is used to verify that the data extracted from the EMR and document repository is both necessary and sufficient to answer all form items and medical necessity items. The checklist may be specific to each payor and each procedure in various examples.

The new prior authorization request may be submitted in response to the determining that the payor will be approved by the payor.

If the checklist is not complete, then one of two options is executed, based on the operational parameters of the customer and the quality-plus-quantity of underlying similar data. The first option is to put the worklist item in a queue for review by a person. A typical reason that the checklist cannot be completed is that external data is required but was not accessible by the system. The person may work, via user interface 130, to obtain the data and include it in the submission list by manual upload. Or the person may determine that the information in question is not necessary and override the checklist. The second option is to use algorithmic determination that the payer will approve the request without every checklist item satisfied, based on recent similar cases, and mark the request as ready to be submitted to the payer.

If the worklist item is displayed to a person at this stage, the system displays an estimate of approval success, given recent similar cases with the same payer and the same code. The estimate accounts for the presence of or lack of necessary but complete answers to questions contained in the payer's submission template form as well as any medical necessity questions.

FIG. 4 is a flowchart illustrating an example computer implemented method 400 of determining whether or not the request is likely to be approved by a payor. A set of training and test request, also referred to as packets, is shown at 410. The training requests comprise examples of requests that were approved and are thus labeled. Test packets are not labeled but are known to include prior requests that have been both approved and denied. RML versions of the requests are indicated at shown at 415 and 420 respectively. The training RML requests are provided to a neural network 425 to train a model 440. The model thus learns which requests are mostly likely to be approved. To test the model, test RML requests 420 are provided to the model with the results 445 checked to ensure that the model is accurate. In some examples, each result has an associated confidence level that may be compared to a threshold. The threshold may be set based on many factors, including cost of resubmission versus difficulty and cost of obtaining further data to ensure successful submission.

When either the system or the person using the system determines that the package of info is sufficient for submission to the payer, the system checks the database to determine if the submission can be made using discrete electronic methods or must be sent via fax. If fax, the system formats each unique item of information onto prearranged client letterhead with header and footer as agreed. These pages are then collated, a cover page created automatically, and the bundle of pages faxed to the payer. If the submission can be made electronically, the system determines via database lookup whether the payer requires a “portal” submission or can receive information via an API.

To submit the Prior Authorization package of data to the payer via portal methods, the system breaks the data into discrete responses to the required questions. In one embodiment, Selenium software is used for understanding and navigating payer (insurance company or other paying entity) portals programmatically. As a payer login page is navigated and the first set of questions are revealed, the system feeds the portal the necessary data via Selenium functions. The system utilizes algorithms to analyze the next set of questions as identified by Selenium, and provide contextual answers based on the questions themselves. In this method, any modifications to the portal pages will be automatically handled without programmatic intervention.

To submit the Prior Authorization package of data to the payer via API, the system implements the API calls and process responses using the payer's documented API interface.

After submission, the system monitors the payer's status regarding processing the submission and providing a response. A response of Approved requires no further interaction, and the response details are pushed to the EMR by the system. Other responses may require further action. Further action is initiated by placing the worklist item into a queue for a person to open and resolve, as indicated by a status determined by the payer's response.

Some payer responses dictate a range of dates for when the order is approved, including an end-date. Some payer responses dictate that the order, if recurring, must have the Prior Authorization repeated periodically, often from scratch. Some payer responses dictate that the location of the procedure or device delivery must be at a specific location, as opposed to wherever the physician determines most convenient or medically relevant. In these cases, the system displays the payer's response to the person, inserts the response into the EMR, and the person follows through with whatever next action is determined appropriate.

In one embodiment, the system may also operate within the payer. Initial implementation includes an ingest of several years of claims and PA data, used to train the ML (Machine Learning) algorithms. These algorithms are tuned and biased to represent current states of policy and requirements, but also are annealed to understand that recent historic decisions could vary from the then-current-published policies and requirements. This annealment allows the system to judge a Prior Authorization request in a manner similar to a person.

To evaluate a Prior Authorization request at a payer, the system first assess the potential of the submission to be processed by the system. If the submission was submitted via fax, the pages are sent through OCR and NLP algorithms to enable the data representation to be processed. The system then uses the same subsequent algorithms to determine if the submission meets all necessary information and medical necessity requirements, as specified by the payer's policies.

The system processes a PA request at a payor in the same manner as an inspection process that is used on any manufacturing or repetitive high-volume task. The requirements for grading the submission are listed by the payer. The system compares the content of the request against the requirements. The result is a grade of pass or fail. If the grade is fail the submission is transferred to a queue for a person to review and pursue further.

For submissions that were submitted by the system in use by the provider organization, the system already knows that the submitted data matches the payer requirements. If the submission does not match the payer requirements, the system at the provider would have flagged the worklist item as a low probability of being approved and required a person to evaluate the issues and resolve the missing or incorrect data. Clean submissions are therefore automatically approved. Submissions that contain gaps or missing items are placed into a queue for a person to review and judge.

In some or all implementations of some or all client categories, the system may provide reports and dashboards of data analytics including staff productivity, payer and provider responses, ordering and prescribing trends, and any other custom KPI as may be represented by the data within the database.

Artificial intelligence (AI) is a field concerned with developing decision-making systems to perform cognitive tasks that have traditionally required a living actor, such as a person. Artificial neural networks (ANNs) are computational structures that are loosely modeled on biological neurons. Generally, ANNs encode information (e.g., data or decision making) via weighted connections (e.g., synapses) between nodes (e.g., neurons). Modern ANNs are foundational to many AI applications, such as automated perception (e.g., computer vision, speech recognition, contextual awareness, etc.), automated cognition (e.g., decision-making, logistics, routing, supply chain optimization, etc.), automated control (e.g., autonomous cars, drones, robots, etc.), among others.

Many ANNs are represented as matrices of weights that correspond to the modeled connections. ANNs operate by accepting data into a set of input neurons that often have many outgoing connections to other neurons. At each traversal between neurons, the corresponding weight modifies the input and is tested against a threshold at the destination neuron. If the weighted value exceeds the threshold, the value is again weighted, or transformed through a nonlinear function, and transmitted to another neuron further down the ANN graph—if the threshold is not exceeded then, generally, the value is not transmitted to a down-graph neuron and the synaptic connection remains inactive. The process of weighting and testing continues until an output neuron is reached; the pattern and values of the output neurons constituting the result of the ANN processing.

The correct operation of most ANNs relies on correct weights. However, ANN designers do not generally know which weights will work for a given application. Instead, a training process is used to arrive at appropriate weights. ANN designers typically choose a number of neuron layers or specific connections between layers including circular connection, but the ANN designer does not generally know which weights will work for a given application. Instead, a training process generally proceeds by selecting initial weights, which may be randomly selected. Training data is fed into the ANN and results are compared to an objective function that provides an indication of error. The error indication is a measure of how wrong the ANN's result was compared to an expected result. This error is then used to correct the weights. Over many iterations, the weights will collectively converge to encode the operational data into the ANN. This process may be called an optimization of the objective function (e.g., a cost or loss function), whereby the cost or loss is minimized.

A gradient descent technique is often used to perform the objective function optimization. A gradient (e.g., partial derivative) is computed with respect to layer parameters (e.g., aspects of the weight) to provide a direction, and possibly a degree, of correction, but does not result in a single correction to set the weight to a “correct” value. That is, via several iterations, the weight will move towards the “correct,” or operationally useful, value. In some implementations, the amount, or step size, of movement is fixed (e.g., the same from iteration to iteration). Small step sizes tend to take a long time to converge, whereas large step sizes may oscillate around the correct value or exhibit other undesirable behavior. Variable step sizes may be attempted to provide faster convergence without the downsides of large step sizes.

Backpropagation is a technique whereby training data is fed forward through the ANN—here “forward” means that the data starts at the input neurons and follows the directed graph of neuron connections until the output neurons are reached—and the objective function is applied backwards through the ANN to correct the synapse weights. At each step in the backpropagation process, the result of the previous step is used to correct a weight. Thus, the result of the output neuron correction is applied to a neuron that connects to the output neuron, and so forth until the input neurons are reached. Backpropagation has become a popular technique to train a variety of ANNs.

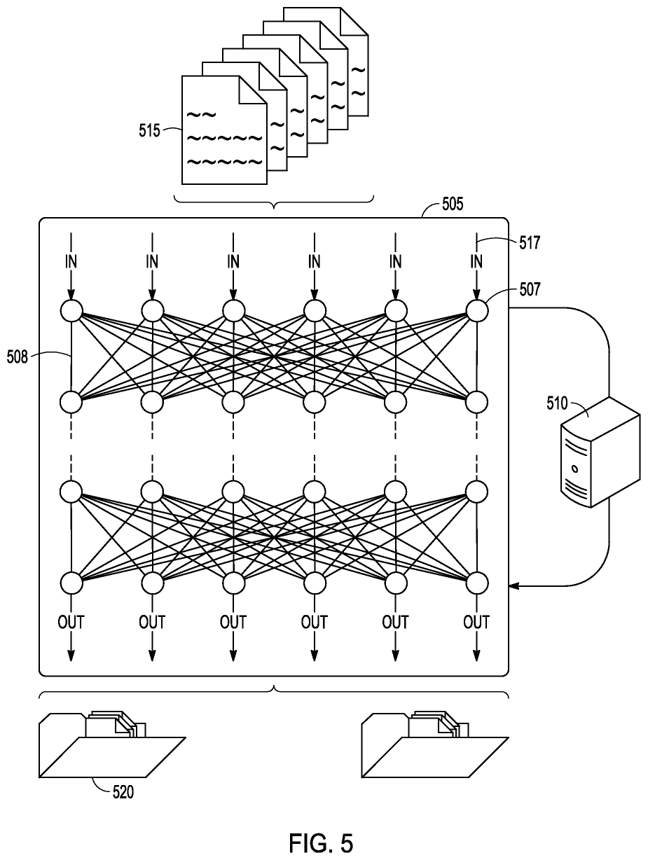

FIG. 5 is a block diagram of an example of an environment including a system for neural network training for implementing one or more machine learning algorithms, according to an embodiment. The system includes an ANN 505 that is trained using a processing node 510. The processing node 510 may be a CPU, GPU, field programmable gate array (FPGA), digital signal processor (DSP), application specific integrated circuit (ASIC), or other processing circuitry. In an example, multiple processing nodes may be employed to train different layers of the ANN 505, or even different nodes 507 within layers. Thus, a set of processing nodes 510 is arranged to perform the training of the ANN 505.

The set of processing nodes 510 is arranged to receive a training set 515 for the ANN 505. The ANN 505 comprises a set of nodes 507 arranged in layers (illustrated as rows of nodes 507) and a set of inter-node weights 508 (e.g., parameters) between nodes in the set of nodes. In an example, the training set 515 is a subset of a complete training set. Here, the subset may enable processing nodes with limited storage resources to participate in training the ANN 505.

The training data may include multiple numerical values representative of a domain, such as red, green, and blue pixel values and intensity values for an image or pitch and volume values at discrete times for speech recognition. Each value of the training or input 517 to be classified once ANN 505 is trained, is provided to a corresponding node 507 in the first layer or input layer of ANN 505. The values propagate through the layers and are changed by the objective function.

As noted above, the set of processing nodes is arranged to train the neural network to create a trained neural network. Once trained, data input into the ANN will produce valid classifications 520 (e.g., the input data 517 will be assigned into categories), for example. The training performed by the set of processing nodes 507 is iterative. In an example, each iteration of the training the neural network is performed independently between layers of the ANN 505. Thus, two distinct layers may be processed in parallel by different members of the set of processing nodes. In an example, different layers of the ANN 505 are trained on different hardware. The members of different members of the set of processing nodes may be located in different packages, housings, computers, cloud-based resources, etc. In an example, each iteration of the training is performed independently between nodes in the set of nodes. This example is an additional parallelization whereby individual nodes 507 (e.g., neurons) are trained independently. In an example, the nodes are trained on different hardware.

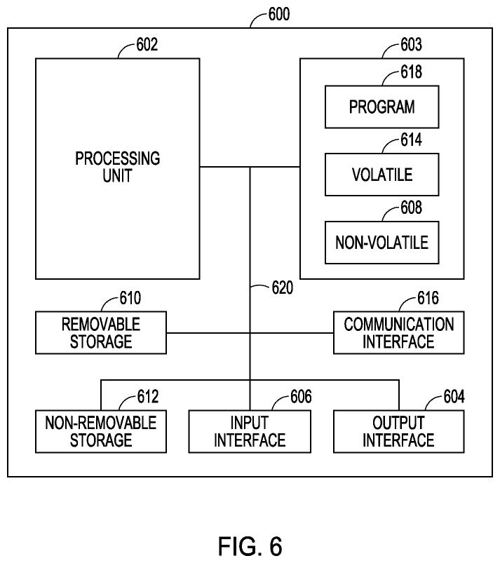

FIG. 6 is a block schematic diagram of a computer system 600 to implement and manage the use of flexible workspaces and for performing methods and algorithms according to example embodiments. All components need not be used in various embodiments.

One example computing device in the form of a computer 600 may include a processing unit 602, memory 603, removable storage 610, and non-removable storage 612. Although the example computing device is illustrated and described as computer 600, the computing device may be in different forms in different embodiments. For example, the computing device may instead be a smartphone, a tablet, smartwatch, smart storage device (SSD), or other computing device including the same or similar elements as illustrated and described with regard to FIG. 6. Devices, such as smartphones, tablets, and smartwatches, are generally collectively referred to as mobile devices or user equipment.

Although the various data storage elements are illustrated as part of the computer 600, the storage may also or alternatively include cloud-based storage accessible via a network, such as the Internet or server-based storage. Note also that an SSD may include a processor on which the parser may be run, allowing transfer of parsed, filtered data through I/O channels between the SSD and main memory.

Memory 603 may include volatile memory 614 and non-volatile memory 608. Computer 600 may include—or have access to a computing environment that includes—a variety of computer-readable media, such as volatile memory 614 and non-volatile memory 608, removable storage 610 and non-removable storage 612. Computer storage includes random access memory (RAM), read only memory (ROM), erasable programmable read-only memory (EPROM) or electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions.

Computer 600 may include or have access to a computing environment that includes input interface 606, output interface 604, and a communication interface 616. Output interface 604 may include a display device, such as a touchscreen, that also may serve as an input device. The input interface 606 may include one or more of a touchscreen, touchpad, mouse, keyboard, camera, one or more device-specific buttons, one or more sensors integrated within or coupled via wired or wireless data connections to the computer 600, and other input devices. The computer may operate in a networked environment using a communication connection to connect to one or more remote computers, such as database servers. The remote computer may include a personal computer (PC), server, router, network PC, a peer device or other common data flow network switch, or the like. The communication connection may include a Local Area Network (LAN), a Wide Area Network (WAN), cellular, Wi-Fi, Bluetooth, or other networks. According to one embodiment, the various components of computer 600 are connected with a system bus 620.

Computer-readable instructions stored on a computer-readable medium are executable by the processing unit 602 of the computer 600, such as a program 618. The program 618 in some embodiments comprises software to implement one or more methods when executing on one or more processors of processing unit 602. A hard drive, CD-ROM, and RAM are some examples of articles including a non-transitory computer-readable medium such as a storage device. The terms computer-readable medium and storage device do not include carrier waves to the extent carrier waves are deemed too transitory. Storage can also include networked storage, such as a storage area network (SAN). Computer program 618 along with the workspace manager 622 may be used to cause processing unit 602 to perform one or more methods or algorithms described herein.

Although a few embodiments have been described in detail above, other modifications are possible. For example, the logic flows depicted in the figures do not require the particular order shown, or sequential order, to achieve desirable results. Other steps may be provided, or steps may be eliminated, from the described flows, and other components may be added to, or removed from, the described systems. Other embodiments may be within the scope of the following claims.

1. A computer implemented method comprising:
receiving an indication of a medical order for a patient;
accessing insurance information of the patient, such insurance information identifying a payor;
accessing an electronic medical record corresponding to the patient;
using a machine learning program trained on properly filled out payor specific prior authorization requests to generate a new prior authorization request corresponding to the medical order based on data in the electronic medical record; and
submitting the generated new prior authorization request to the payor for processing.

2. The method of claim 1 and further comprising querying the electronic medical record for the new prior authorization request specific information.

3. The method of any of claim 1 and further comprising performing optical character recognition on electronic medical record data not in text form.

4. The method of claim 1 wherein medical orders are received by polling new orders via a pull message or receiving push messages corresponding to medical orders.

5. The method of claim 1 and further comprising querying the payor to verify patient coverage.

6. The method of claim 1 and further comprising using a combination of fuzzy logic and machine learning to determine a date or date range from the medical order to provide with the new prior authorization request.

7. The method of claim 1 and further comprising using a combination of fuzzy logic, contextual understanding, and anticipation of wayward behavior built within compact AI and NLP (Natural Language Processing) algorithms to address wrong field placement of data in the electronic medical record.

8. The method of claim 1 and further comprising:
using a checklist identifying data items for generating the new prior authorization request;
determining data items in the checklist not satisfied;
determining that the payor will approve the new prior authorization request without the data items determined to be not satisfied; and
submitting the new prior authorization request in response to the determining that the payor will approve.

9. The method of claim 1 wherein the new prior authorization request is submitted in a format and manor specified by the payor.

10. A machine-readable storage device having instructions for execution by a processor of a machine to cause the processor to perform operations to perform a method, the operations comprising:
receiving an indication of a medical order for a patient;
accessing insurance information of the patient, such insurance information identifying a payor;
accessing an electronic medical record corresponding to the patient;
using a machine learning program trained on properly filled out payor specific prior authorization requests to generate a new prior authorization request corresponding to the medical order based on data in the electronic medical record; and
submitting the generated new prior authorization request to the payor for processing.

11. The device of claim 10 wherein the operations further comprise querying the electronic medical record for authorization new prior authorization request specific information.

12. The device of claim 10 wherein the operations further comprise performing optical character recognition on electronic medical record data not in text form.

13. The device of claim 10 wherein medical orders are received by polling new orders via a pull message or receiving push messages corresponding to medical orders.

14. The device of claim 10 wherein the operations further comprise querying the payor to verify patient coverage.

15. The device of claim 10 wherein the operations further comprise using a combination of fuzzy logic and machine learning to determine a date or date range from the medical order to provide with the new prior authorization request.

16. The device of claim 10 wherein the operations further comprise using a combination of fuzzy logic, contextual understanding, and anticipation of wayward behavior built within compact AI and NLP (Natural Language Processing) algorithms to address wrong field placement of data in the electronic medical record.

17. The device of claim 10 wherein the operations further comprise:
using a checklist identifying data items for generating the new prior authorization request;
determining data items in the checklist not satisfied;
determining that the payor will approve the new prior authorization request without the data items determined to be not satisfied; and
submitting the new prior authorization request in response to the determining that the payor will approve.

18. A device comprising:
a processor; and
a memory device coupled to the processor and having a program stored thereon for execution by the processor to perform operations comprising:
receiving an indication of a medical order for a patient;
accessing insurance information of the patient, such insurance information identifying a payor;
accessing an electronic medical record corresponding to the patient;
using a machine learning program trained on properly filled out payor specific prior authorization requests to generate a new prior authorization request corresponding to the medical order based on data in the electronic medical record; and
submitting the generated new prior authorization request to the payor for processing.

19. The device of claim 18 wherein the operations further comprise:
querying the electronic medical record for new prior authorization request specific information;
querying the payor to verify patient coverage;
using a machine learning trained model to determine a date or date range from the medical order to provide with the new prior authorization request;
using a checklist identifying data items for generating the new prior authorization request;
determining data items in the checklist not satisfied;
determining that the payor will approve the new prior authorization request without the data items determined to be not satisfied; and
submitting the request in response to the determining that the payor will approve.

20. The device of claim 18 wherein the operations further comprise performing optical character recognition on electronic medical record data not in text form and wherein medical orders are received by polling new orders via a pull message or receiving push messages corresponding to medical orders.
